SEQUENCE LISTING (37 sequences)

SEQ ID NO: 1 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 2 - protein, 482 aa, Homo sapiens
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 3 - protein, 653 aa, Homo sapiens
MRPLRPRAALLALLASLLAAPPVAPAEAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRGPPSPGNP

SEQ ID NO: 4 - protein, 626 aa, Homo sapiens
APHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 5 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 6 - protein, 517 aa, Homo sapiens
TDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 7 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 8 - protein, 627 aa, Homo sapiens
EAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRGPPSPGNP

SEQ ID NO: 9 - protein, 617 aa, Homo sapiens
XAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPV

SEQ ID NO: 10 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 11 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 12 - protein, 525 aa, Homo sapiens; identical to SEQ ID NO: 7

SEQ ID NO: 13 - protein, 525 aa, Homo sapiens
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 14 - protein, 714 aa, synthetic construct
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 713 aa, synthetic construct
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 715 aa, synthetic construct
RPRNALLLLADDGGFESGAYNNSAIATPHLDALARRSLLFRNAFTSVSSFGSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 714 aa, synthetic construct
RPRNALLLLADDGGFESGAYNNSAIATPHLDALARRSLLFRNAFTSVSSFGSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 714 aa, synthetic construct
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 19 - protein, 713 aa, synthetic construct
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 20 - protein, 715 aa, synthetic construct
RPRNALLLLADDGGFESGAYNNSAIATPHLDALARRSLLFRNAFTSVSSFGSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 21 - protein, 714 aa, synthetic construct
RPRNALLLLADDGGFESGAYNNSAIATPHLDALARRSLLFRNAFTSVSSFGSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 22 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 23 - protein, 226 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 24 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 26 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 27 - protein, 127 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYK

SEQ ID NO: 28 - protein, 113 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIW

SEQ ID NO: 29 - protein, 115 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPS

SEQ ID NO: 30 - protein, 178 aa, Homo sapiens
LIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQISVHHCARAARKVAHFPGYTTHPQESLSCCFLVGYHREALPQRQFVQ

SEQ ID NO: 31 - protein, 333 aa, Homo sapiens
MVSKSRWKLLAMLALVLVVMVWYSISREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 32 - protein, 332 aa, Homo sapiens
MCPAGWKLLAMLALVLVVMVWYSISREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 33 - protein, 322 aa, Homo sapiens
MLALVLVVMVWYSISREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 34 - protein, 339 aa, Homo sapiens
MVSKSRWKLLAMLALVLVVMVWYSISREDRYIELFYFPIPEKKEPCLQGEAESKASKLFGKLSPLCSYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 35 - protein, 329 aa, Homo sapiens
MVSKSRWKLLAMLALVLVVMVWYSISREDSFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 36 - protein, 328 aa, Homo sapiens
MCPAGWKLLAMLALVLVVMVWYSISREDSFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 37 - protein, 318 aa, Homo sapiens
MLALVLVVMVWYSISREDSFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

(19) United States
(12) Patent Application Publication
(10) Pub. No.: US 2026/0308601 A1
(43) Pub. Date: Oct. 8, 2026

(54) METHODS OF INCREASING SIALIC ACID LEVELS IN RECOMBINANT GLYCOSYLATED PROTEINS

(71) Applicant: DENALI THERAPEUTICS INC., South San Francisco, CA (US)

(72) Inventors: Melina LENSER, South San Francisco, CA (US); Hanh Giai NGO, South San Francisco, CA (US); Anh Thuy NGUYEN DANG, South San Francisco, CA (US); Allain Erfe PETERSEN, South San Francisco, CA (US); Yashas RAJENDRA, South San Francisco, CA (US)

(21) Appl. No.: 19/476,827

(22) PCT Filed: Apr. 22, 2024
(86) PCT No.: PCT/US2024/025699

§ 371 Date: Oct. 20, 2025

Related U.S. Application Data
(60) Provisional application No. 63/461,211, filed on Apr. 21, 2023.

(51) Int. Cl. C12N 15/62; A61K 38/17; C12N 9/10; C12N 9/24; C12P 21/00

(52) U.S. Cl. CPC C12N 15/62; A61K 38/1741; C12N 9/1051; C12N 9/2402; C12P 21/005; C12Y 302/01076; C12Y 310/01001

(57) ABSTRACT

Provided herein are methods of increasing the sialic acid level in a recombinant glycosylated protein, recombinant glycosylated proteins produced by these methods, and methods of treating a subject in need thereof using these recombinant glycosylated proteins.

### CROSS-REFERENCE TO RELATED APPLICATIONS

This application claims priority to U.S. Provisional Patent Application No. 63/461,211, filed Apr. 21, 2023; the entire contents of which are herein incorporated by reference.

### SEQUENCE LISTING

This application contains a Sequence Listing that has been submitted electronically as an XML file named 47364-0050WO1_ST26_SL The XML file, created on Apr. 12, 2024, is 1,210,951 bytes in size. The material in the XML file is hereby incorporated by reference in its entirety.

### TECHNICAL FIELD

This disclosure relates to methods of cell culturing and the biomanufacturing of recombinant glycosylated proteins.

### BACKGROUND

Lysosomal storage disorders (LSDs) are relatively rare, inherited metabolic diseases that result from defects in lysosomal function. LSDs are typically caused by the deficiency of a single enzyme that participates in the breakdown of metabolic products in the lysosome. The buildup of the product resulting from lack of the enzymatic activity affects various organ systems and can lead to severe symptoms and premature death. The majority of LSDs also have a significant neurological component, which ranges from progressive neurodegeneration and severe cognitive impairment to epileptic, behavioral, and psychiatric disorders. A recombinant form of an enzyme that is deficient in an LSD can be used to treat the disorder.

LSD enzymes form part of a group of glycosylated proteins which, when produced in cell culture, can exhibit heterogeneity that is partly due to the glycosylation pattern of the protein. Glycosylation patterns can be strongly influenced by cell culture conditions. In turn, the glycosylation (including galactosylation) profile of a particular protein can influence its biological activity due to variable effects on folding, stability, efficacy, and half-life. Various factors in cell culture are linked, such that one factor (e.g., adding galactose to media) may on the one hand favorably influence the glycosylation pattern of the recombinant protein but may, on the other hand, also have negative effects on other characteristics of the protein, such as the charge profile, proportion of protein fragments, proportion of aggregates, and protein titer. Furthermore, cell viability may be affected. Accordingly, there is a need for methods that improve glycosylation patterns, such as improving sialic acid content, in recombinantly-produced glycosylated proteins.

### SUMMARY

Provided herein are methods of increasing the sialic acid level in a recombinant glycosylated protein that includes: culturing a mammalian host cell capable of expressing the recombinant glycosylated protein in a liquid culture medium including one or more media additives selected from the group consisting of: hydrocortisone, N-acetylmannosamine (ManNAc), manganese, and uridine, wherein the mammalian host cell has been previously transformed with one or more vectors encoding one or more sialyltransferases.

In some embodiments of any of the methods described herein, the method further includes, prior to the culturing step, transforming the mammalian cell capable of expressing the recombinant glycosylated protein with one or more vectors encoding the one or more sialyltransferases to generate the mammalian host cell. In some embodiments, the mammalian host cell is a rodent cell. In some embodiments, the rodent cell is a CHO cell. In some embodiments of any of the methods described herein, the mammalian host cell is a human cell. In some embodiments of any of the methods described herein, the one or more sialyltransferases are selected from the group consisting of: ST6GAL1 and ST3GAL4. In some embodiments, one of the one or more vectors encodes ST6GAL1.

In some embodiments of any of the methods described herein, the liquid culture medium includes one of: hydrocortisone, N-acetylmannosamine (ManNAc), manganese, or uridine. In some embodiments of any of the methods described herein, the liquid culture medium includes two of: hydrocortisone, N-acetylmannosamine (ManNAc), manganese, and uridine. In some embodiments of any of the methods described herein, the liquid culture medium includes three of: hydrocortisone, N-acetylmannosamine (ManNAc), manganese, and uridine. In some embodiments of any of the methods described herein, the liquid culture medium includes hydrocortisone, N-acetylmannosamine (ManNAc), manganese, and uridine. In some embodiments of any of the methods described herein, the ManNAc is present in the liquid culture medium at a concentration of from 10 mM to about 60 mM. In some embodiments of any of the methods described herein, the uridine is present in the liquid culture medium at a concentration of from 2.5 mM to about 10 mM. In some embodiments of any of the methods described herein, the hydrocortisone is present in the liquid culture medium at a concentration of from 5 μM to about 50 μM. In some embodiments of any of the methods described herein, the manganese is present in the liquid culture medium at a concentration of from 2 μM to about 15 μM.

In some embodiments of any of the methods described herein, the recombinant glycosylated protein is a recombinant glycosylated enzyme. In some embodiments, the recombinant glycosylated enzyme includes an enzyme replacement therapy (ERT) enzyme, a catalytically active ERT enzyme variant, or a catalytically active ERT enzyme fragment. In some embodiments, the enzyme replacement therapy (ERT) enzyme includes a lyosomal storage disease (LSD) enzyme. In some embodiments, the LSD enzyme is alpha-L-iduronidase (IDUA) or N-sulfoglucosamine sulfohydrolase (SGSH). In some embodiments, the recombinant glycosylated enzyme is a fusion protein. In some embodiments, the fusion protein includes (i) a first Fc polypeptide that is linked to an enzyme replacement therapy (ERT) enzyme, a catalytically active ERT enzyme variant, or a catalytically active ERT enzyme fragment; and (ii) a second Fc polypeptide that forms an Fc dimer with the first Fc polypeptide, wherein the first Fc polypeptide and/or the second Fc polypeptide is a modified Fc polypeptide capable of specifically binding to a transferrin receptor (TfR). In some embodiments, the fusion protein includes (i) an enzyme replacement therapy (ERT) enzyme, a catalytically active ERT enzyme variant, or a catalytically active ERT enzyme fragment; and (ii) a modified Fc dimer that is capable of specifically binding to a transferrin receptor (TfR). In some embodiments, the fusion protein includes an enzyme replacement therapy (ERT) enzyme, a catalytically active ERT enzyme variant, or a catalytically active ERT enzyme fragment, linked to an Fc polypeptide. In some embodiments, the Fc polypeptide is a modified Fc polypeptide. In some embodiments, the Fc polypeptide is capable of specifically binding to a transferrin receptor (TfR).

In some embodiments of any of the methods described herein, the culturing is fed-batch culturing. In some embodiments of any of the methods described herein, the culturing is batch culturing. In some embodiments of any of the methods described herein, the culturing is perfusion culturing.

In some embodiments of any of the methods described herein, the sialic acid level in the recombinant glycosylated protein is increased by at least 50% as compared to the recombinant glycosylated protein produced by a method that does not include one or both of (i) the use of a mammalian host cell capable of expressing the recombinant glycosylated protein including one or more vectors encoding the one or more sialyltransferases, and (ii) culturing a mammalian host cell capable of expressing the recombinant glycosylated protein in a liquid culture medium including one or more media additives selected from the group consisting of: hydrocortisone, N-acetylmannosamine (ManNAc), manganese, and uridine.

In some embodiments of any of the methods described herein, the sialic acid level in the recombinant glycosylated protein is increased by at least 100% as compared to the recombinant glycosylated protein produced by a method that does not include one or both of (i) the use of a mammalian host cell capable of expressing the recombinant glycosylated protein including one or more vectors encoding the one or more sialyltransferases, and (ii) culturing a mammalian host cell capable of expressing the recombinant glycosylated protein in a liquid culture medium including one or more media additives selected from the group consisting of: hydrocortisone, N-acetylmannosamine (ManNAc), manganese, and uridine.

In some embodiments of any of the methods described herein, the sialic acid level in the recombinant glycosylated protein is increased by at least 150% as compared to the recombinant glycosylated protein produced by a method that does not include one or both of (i) the use of a mammalian host cell capable of expressing the recombinant glycosylated protein including one or more vectors encoding the one or more sialyltransferases, and (ii) culturing a mammalian host cell capable of expressing the recombinant glycosylated protein in a liquid culture medium including one or more media additives selected from the group consisting of: hydrocortisone, N-acetylmannosamine (ManNAc), manganese, and uridine.

In some embodiments of any of the methods described herein, the sialic acid level in the recombinant glycosylated protein is increased by at least 250% as compared to the recombinant glycosylated protein produced by a method that does not include one or both of (i) the use of a mammalian host cell capable of expressing the recombinant glycosylated protein including one or more vectors encoding the one or more sialyltransferases, and (ii) culturing a mammalian host cell capable of expressing the recombinant glycosylated protein in a liquid culture medium including one or more media additives selected from the group consisting of: hydrocortisone, N-acetylmannosamine (ManNAc), manganese, and uridine.

In some embodiments of any of the methods described herein, the method further includes harvesting the recombinant glycosylated protein from the liquid culture medium and/or the mammalian host cell.

In some embodiments of any of the methods described herein, the method further includes isolating the harvested recombinant glycosylated protein.

In some embodiments of any of the methods described herein, the method further includes formulating the isolated recombinant glycosylated protein.

Also provided herein are recombinant glycosylated proteins produced by any of the methods described herein.

Also provided herein are methods of treating a subject in need thereof including administering to the subject a therapeutically effective amount of any of the recombinant glycosylated proteins described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a diagram illustrating key pathways affecting sialylation and areas of evaluation that were tested for recombinant fusion protein production.

FIG. 2 is a bar graph showing the percentage sialic acid improvement for SGSH-Fc fusion proteins produced in the presence of different media additives: 20 μM hydrocortisone, 40 mM ManNAC, 10 μM Mn, or 5 mM uridine.

FIG. 3 is a bar graph that includes an illustration of the percentage sialic acid improvement for a pool (Pool-B) and its clones (Clone B-1 and Clone B-2) expressing IDUA-Fc fusion proteins.

FIG. 4 is a bar graph that includes an illustration of the percentage sialic acid improvement for pools expressing IDUA-Fc proteins under different culture conditions and in the presence or absence of sialyltransferase gene overexpression.

FIG. 5 is a bar graph that includes an illustration of the percentage sialic acid improvement for pools expressing IDUA-Fc proteins under different culture conditions and in the presence or absence of galactosyltransferase an/or sialyltransferase gene overexpression.

### DETAILED DESCRIPTION

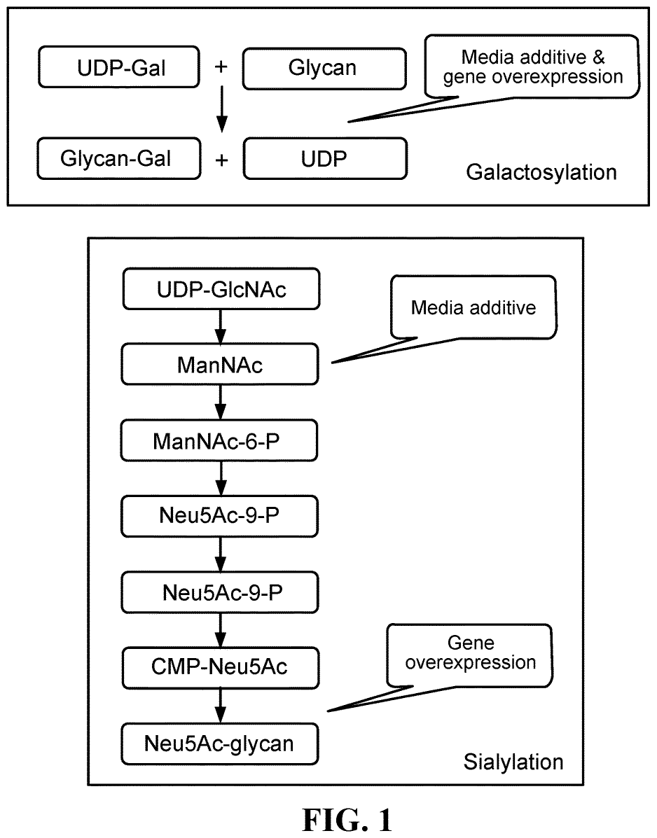

Provided herein are methods of increasing the sialic acid level in a recombinant glycosylated protein that include: culturing a mammalian host cell capable of expressing the recombinant glycosylated protein in a liquid culture medium including one or more (e.g., 1, 2, 3, or 4) media additives selected from the group consisting of: hydrocortisone, N-acetylmannosamine (ManNAc), manganese, and uridine, wherein the mammalian host cell has been previously transformed with one or more vectors encoding one or more sialyltransferases. In some embodiments, the mammalian host cell has also been previously transformed with one or more vectors encoding a galactosyltransferase. FIG. 1 illustrates the metabolic steps affecting sialylation and areas of evaluation that were tested for recombinant fusion protein production.

In some embodiments, the recombinant glycosylated protein is an ERT enzyme, a catalytically active ERT enzyme variant, or a catalytically active ERT enzyme fragment. In some embodiments, the recombinant glycosylated protein is a fusion protein (e.g., any of the exemplary fusion proteins described herein, e.g., a fusion protein comprising an ERT enzyme). Embodiments of the same are disclosed herein

### Definitions

As used herein, the singular forms “a,” “an,” and “the” include plural referents unless the content clearly dictates otherwise. Thus, for example, reference to “a recombinant glycosylated protein” may include two or more such recombinant glycosylated proteins, and the like.

As used herein, the terms “about” and “approximately,” when used to modify an amount specified in a numeric value or range, indicate that the numeric value as well as reasonable deviations from the value known to the skilled person in the art, for example±20%, ±10%, or ±5%, are within the intended meaning of the recited value.

An “enzyme replacement therapy enzyme” or “ERT enzyme” refers to an enzyme that is deficient in a disorder (e.g., a lysosomal storage disorder). Examples of ERT enzymes include “lysosomal storage disorder (LSD) enzymes,” which refer to a class of enzymes that are deficient in a lysosomal storage disorder. A “catalytically active ERT enzyme variant” refers to a functional variant, including allelic and splice variants, of a wild-type ERT enzyme or a catalytically active ERT enzyme fragment, where the ERT enzyme variant has at least 50%, at least 55%, at least 60%, at least 65%, at least 70%, at least 75%, at least 80%, at least 85%, at least 90%, or at least 95% of the activity of the corresponding wild-type ERT enzyme or catalytically active ERT enzyme fragment, e.g., when assayed under identical conditions. A “catalytically active ERT enzyme fragment” of an ERT enzyme refers to a portion of a full-length ERT enzyme or a catalytically active ERT enzyme variant, where the catalytically active fragment has at least 50%, at least 55%, at least 60%, at least 65%, at least 70%, at least 75%, at least 80%, at least 85%, at least 90%, or at least 95% of the activity of the corresponding full-length ERT enzyme or catalytically active ERT enzyme variant, e.g., when assayed under identical conditions.

A “sulfoglucosamine sulfohydrolase,” “N-sulfoglucosamine sulfohydrolase,” or “SGSH” as used herein refers to N-sulfoglucosamine sulfohydrolase (EC 3.10.1.1), which is an enzyme involved in the lysosomal degradation of heparan sulfate. Mutations in this gene are associated with Sanfilippo syndrome A, one type of the lysosomal storage disorder mucopolysaccaridosis III, which results from impaired degradation of heparan sulfate. In some embodiments, SGSH can be a component of a recombinant glycosylated protein that further comprises an Fc polypeptide (e.g., any of the exemplary Fc polypeptides or regions described herein). As used herein, SGSH encompasses catalytically active fragments of SGSH as well as functional variants, including allelic and splice variants, of a wild-type SGSH or a fragment thereof. The sequence of human SGSH is available under UniProt entry P51688 and is encoded by the human SGSH gene at 17q25.3. The full-length sequence is provided as SEQ ID NO: 1. A “mature” SGSH sequence as used herein refers to a form of a polypeptide chain that lacks the signal sequence of the naturally occurring full-length polypeptide chain. The amino acid sequence of a mature human SGSH polypeptide is provided as SEQ ID NO: 2, which corresponds to amino acids 21-502 of the full-length human sequence. The structure of human SGSH has been well-characterized. An illustrative structure is available under PDB accession code 4MHX. Non-human primate SGSH sequences have also been described, including chimpanzee (UniProt entry K7C218). A mouse SGSH sequence is available under Uniprot entry Q9EQ08. An SGSH variant has at least 50%, at least 55%, at least 60%, at least 65%, at least 70%, at least 75%, at least 80%, at least 85%, at least 90%, or at least 95% of the activity of the corresponding wild-type SGSH or fragment thereof, e.g., when assayed under identical conditions. A catalytically active SGSH fragment has at least 50%, at least 55%, at least 60%, at least 65%, at least 70%, at least 75%, at least 80%, at least 85%, at least 90%, or at least 95% of the activity of the corresponding full-length SGSH or variant thereof, e.g., when assayed under identical conditions.

An “alpha-L-iduronidase,” “iduronidase alpha-L,” “L-iduronidase,” “iduronidase,” or “IDUA” as used herein refers to alpha-L-iduronidase (EC 3.2.1.76), which is an enzyme involved in the lysosomal degradation of glycosaminoglycans, such as dermatan sulfate and heparan sulfate. Mutations in the IDUA gene are associated with MSP I, which results from impaired degradation of heparan sulfate and dermatan sulfate. The term “IDUA” or “IDUA enzyme” as used herein, optionally as a component of a protein that comprises an Fc polypeptide, encompasses catalytically active fragments of IDUA as well as functional variants, including allelic and splice variants or fragments thereof. A sequence of human IDUA is available under UniProt entry P35475 and is encoded by the human IDUA gene at 4p16.3. A full-length sequence is provided as SEQ ID NO: 3, which may have an H or Q at position 33 and/or an A or T at position 622, wherein the positions are according to EU numbering. A “mature” IDUA sequence as used herein refers to a form of a polypeptide chain that lacks the signal sequence of the naturally occurring full-length polypeptide chain. An embodiment of an amino acid sequence of a mature human IDUA polypeptide is provided as SEQ ID NO: 8, which corresponds to amino acids 27-653 of the full-length human sequence. A “truncated” IDUA sequence as used herein refers to a catalytically active fragment of the naturally occurring full-length polypeptide chain. SEQ ID NO: 9 is an exemplary truncated IDUA sequence. The structure of human IDUA has been well-characterized. Non-human primate IDUA sequences have also been described, including chimpanzee (e.g., UniProt entry A0A2R9ALZ1 for Pan paniscus (Pygmy chimpanzee) (Bonobo)). A mouse IDUA sequence is available under Uniprot entry P48441. An IDUA variant has at least 50%, at least 55%, at least 60%, at least 65%, at least 70%, at least 75%, at least 80%, at least 85%, at least 90%, or at least 95% of the activity of the corresponding wild-type IDUA or fragment thereof, e.g., when assayed under identical conditions. A catalytically active IDUA fragment has at least 50%, at least 55%, at least 60%, at least 65%, at least 70%, at least 75%, at least 80%, at least 85%, at least 90%, or at least 95% of the activity of the corresponding full-length IDUA or variant thereof, e.g., when assayed under identical conditions.

An “iduronate sulfatase,” “iduronate-2-sulfatase,” or “IDS” as used herein refers to iduronate 2-sulfatase (EC 3.1.6.13), which is an enzyme involved in the lysosomal degradation of the glycosaminoglycans heparan sulfate and dermatan sulfate. Deficiency of IDS is associated with Mucopolysaccharidosis II, also known as Hunter syndrome. In some embodiments, IDS can be a component of a recombinant glycosylated protein that further comprises an Fc polypeptide (e.g., any of the exemplary Fc polypeptides or regions described herein). As used herein, IDS encompasses catalytically active fragments of IDS and functional variants, including allelic and splice variants, of a wild-type IDS or a fragment thereof. The sequence of human IDS isoform I, which is the human sequence designated as the canonical sequence, is available under UniProt entry P22304 and is encoded by the human IDS gene at Xq28. The full-length sequence is provided as SEQ ID NO: 5. A “mature” IDS sequence as used herein refers to a form of a polypeptide chain that lacks the signal and propeptide sequences of the naturally occurring full-length polypeptide chain. The amino acid sequence of a mature human IDS polypeptide is provided as SEQ ID NO: 6, which corresponds to amino acids 34-550 of the full-length human sequence. A “truncated” IDS sequence as used herein refers to a catalytically active fragment of the naturally occurring full-length polypeptide chain. The amino acid sequence of an exemplary truncated human IDS polypeptide is provided as SEQ ID NO: 7, which corresponds to amino acids 26-550 of the full-length human sequence. The structure of human IDS has been well-characterized. An illustrative structure is available under PDB accession code 5FQL. The structure is also described in Nat. Comm. 8:15786 doi: 10.1038/ncomms15786, 2017. Non-human primate IDS sequences have also been described, including chimpanzee (UniProt entry K7BKV4) and rhesus macaque (UniProt entry H9FTX2). A mouse IDS sequence is available under Uniprot entry Q08890. An IDS variant has at least 50%, at least 55%, at least 60%, at least 65%, at least 70%, at least 75%, at least 80%, at least 85%, at least 90%, or at least 95% of the activity of the corresponding wild-type IDS or fragment thereof, e.g., when assayed under identical conditions. A catalytically active IDS fragment has at least 50%, at least 55%, at least 60%, at least 65%, at least 70%, at least 75%, at least 80%, at least 85%, at least 90%, or at least 95% of the activity of the corresponding full-length IDS or variant thereof, e.g., when assayed under identical conditions.

A “transferrin receptor” or “TfR” as used herein refers to transferrin receptor protein 1. The human transferrin receptor 1 polypeptide sequence is set forth in SEQ ID NO: 10. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO: 11.

A “[ERT enzyme] fusion polypeptide” refers to a polypeptide that is linked (e.g., fused) to an ERT enzyme, a catalytically active ERT enzyme variant, or a catalytically active fragment thereof. The polypeptide may be linked to the ERT enzyme, catalytically active ERT enzyme variant, or catalytically active ERT enzyme fragment by a peptide bond or by a polypeptide linker. The fusion polypeptide may be an [ERT enzyme]-Fc fusion polypeptide, an [ERT enzyme]-antigen binding domain fusion polypeptide, an [ERT enzyme]-antibody heavy chain fusion polypeptide, an [ERT enzyme]-antibody heavy chain variable region fusion polypeptide, an [ERT enzyme]-antibody light chain fusion polypeptide, an [ERT enzyme]-antibody light chain variable region fusion polypeptide, an [ERT enzyme]-antibody fragment fusion polypeptide, and the like.

A “[ERT enzyme]-Fc fusion polypeptide” as used herein refers to an Fc polypeptide that is linked (e.g., fused) to an ERT enzyme, a catalytically active ERT enzyme variant, or a catalytically active ERT enzyme fragment. The Fc polypeptide may be linked to the ERT enzyme, catalytically active ERT enzyme variant, or catalytically active ERT enzyme fragment by a peptide bond or by a polypeptide linker. The Fc polypeptide may be a modified Fc polypeptide that contains one or more modifications that promote its heterodimerization to another Fc polypeptide. The Fc polypeptide may be a modified Fc polypeptide that contains one or more modifications that confer binding to a transferrin receptor. The Fc polypeptide may be a modified Fc polypeptide that contains one or more modifications that reduce effector function. The Fc polypeptide may be a modified Fc polypeptide that contains one or more modifications that extend serum half-life. Exemplary modified Fc polypeptides are described, for example, in WO 2019/070577.

In some embodiments, a [ERT enzyme]-Fc fusion protein can be a dimeric protein comprising a first Fc polypeptide that is linked (e.g., fused) to an ERT enzyme, a catalytically active ERT enzyme variant, or a catalytically active ERT enzyme fragment (i.e., an “[ERT]-Fc fusion polypeptide”); and a second Fc polypeptide that forms an Fc dimer with the first Fc polypeptide. The second Fc polypeptide may also be linked (e.g., fused) to an ERT enzyme, a catalytically active ERT enzyme variant, or a catalytically active ERT enzyme fragment. The first Fc polypeptide and/or the second Fc polypeptide may be linked to the ERT enzyme, catalytically active ERT enzyme variant, or catalytically active ERT enzyme fragment by a peptide bond or by a polypeptide linker. The first Fc polypeptide and/or the second Fc polypeptide may be a modified Fc polypeptide that contains one or more modifications that promote its heterodimerization to the other Fc polypeptide. The first Fc polypeptide and/or the second Fc polypeptide may be a modified Fc polypeptide that contains one or more modifications that confer binding to a transferrin receptor. The first Fc polypeptide and/or the second Fc polypeptide may be a modified Fc polypeptide that contains one or more modifications that reduce effector function. The first Fc polypeptide and/or the second Fc polypeptide may be a modified Fc polypeptide that contains one or more modifications that extend serum half-life. Exemplary modified Fc polypeptides are described, for example, in WO 2019/070577.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free-OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO: 1 when optimally aligned to SEQ ID NO:1. The polypeptide that is aligned to the reference sequence need not be the same length as the reference sequence.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (ka, time-1) divided by the association rate constant (ka, time-1 M-1). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

As used herein, the term “specifically binds” or “selectively binds” to a target, e.g., TfR, when referring to an engineered TfR-binding polypeptide, TfR-binding peptide, or TfR-binding antibody as described herein, refers to a binding reaction whereby the engineered TfR-binding polypeptide, TfR-binding peptide, or TfR-binding antibody binds to the target with greater affinity, greater avidity, and/or greater duration than it binds to a structurally different target. In typical embodiments, the engineered TfR-binding polypeptide, TfR-binding peptide, or TfR-binding antibody has at least 5-fold, 10-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, an engineered TfR-binding polypeptide, TfR-binding peptide, or TfR-binding antibody specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding antibody may bind exclusively to a human TfR.

The terms “treatment,” “treating,” and the like are used herein to generally mean obtaining a desired pharmacologic and/or physiologic effect. “Treating” or “treatment” may refer to any indicia of success in the treatment or amelioration of a lysosomal storage disorder, e.g., mucopolysaccharidosis type I, Hunter syndrome (also referred to as “mucopolysaccharidosis type II” or “MPS II”), Sanfilippo syndrome A (also referred to as “mucopolysaccharidosis type IIIA,” “MPS IIIA,” or “Sanfilippo syndrome Type A”), Niemann-Pick disease, Pompe Disease, Gaucher's disease, or Parkinson's disease, including any objective or subjective parameter such as abatement, remission, improvement in patient survival, increase in survival time or rate, diminishing of symptoms or making the disorder more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount,” “therapeutically effective amount,” or “therapeutically effective concentration” of an agent is an amount or concentration of the agent that treats signs or symptoms of a disease (e.g., an LSD) in the subject (e.g., mammal).

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the polypeptides described herein are administered intravenously.

The term “expression” or “expresses” are used herein to refer to transcription and translation occurring within a host cell. Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “cell culture medium” as used herein refers to any cell culture medium used to culture cells that has not been modified either by supplementation, or by selective removal of a certain component.

### Nucleic Acids, Vectors, and Host Cells

The methods described herein use mammalian host cells to produce recombinant glycosylated proteins. Accordingly, the present disclosure provides isolated nucleic acids comprising a nucleic acid sequence encoding any of the recombinant glycosylated proteins described herein and vectors comprising such a nucleic acid, and mammalian host cells into which the nucleic acid/nucleic acids (or vector/vectors) has been introduced that are used to replicate the recombinant glycosylated protein-encoding nucleic acids and/or to express the recombinant glycosylated proteins.

Also provided herein are isolated nucleic acids comprising a nucleic acid sequence encoding one or more sialyltransferases (e.g., any of the sialyltransferases described herein) and vectors comprising such a nucleic acid, and mammalian host cells into which the nucleic acid/nucleic acids (or vector/vectors) has been introduced that are used to replicate the sialyltransferase-encoding nucleic acids and/or to express the one or more sialyltransferases.

Also provided herein are mammalian host cells that comprise (1) a nucleic acid encoding the recombinant glycosylated protein (e.g., any of the recombinant glycosylated proteins described herein) or a vector comprising such a nucleic acid, and (2) one or more nucleic acids encoding one or more sialyltransferases (e.g., any of the exemplary sialyltransferases described herein or known in the art) or one or more vectors comprising such nucleic acid/nucleic acids. In some embodiments, the mammalian host cells further comprise (3) a nucleic acid encoding a galactosyltransferase (e.g., a galactosyltransferase as described herein or known in the art).

In some embodiments, the one or more nucleic acids encoding the one or more sialyltransferases are selected from the group consisting of: ST6GAL1 and ST3GAL4. In some embodiments, the ST6GAL1 a human ST6GAL1 (e.g., NCBI Accession No. NP_001340845.1). In some embodiments, the ST3GAL4 is a human ST3GAL4 (e.g., NCBI Accession No. XP_047283377.1) or a CHO ST3GAL4 (e.g., NCBI Accession No. NP_001233628.1).

In some embodiments, the galactosyltransferase is B4GALT1. In some embodiments, the B4GALT1 is a human B4GALT1 (e.g., NCBI Accession No. NP_001488.2) or a CHO B4GALT1 (e.g., NCBI Accession No. NP_001233620.1).

In some embodiments, the mammalian host cell is a rodent cell (e.g., a Chinese hamster ovary cell) or a human cell.

In some embodiments, the nucleic acids provided herein may be single-stranded or double-stranded. In some embodiments, the nucleic acid is DNA. In particular embodiments, the nucleic acid is cDNA. In some embodiments, the nucleic acid is RNA.

In some embodiments, the vector or vectors can be selected from a plasmid, a viral vector, a phagemid, a yeast chromosomal vector, and a non-episomal mammalian vector. In some embodiments, the vector or vectors can be a transposon.

In some embodiments, the nucleic acid is operably linked to one or more regulatory nucleotide sequences in an expression construct.

Exemplary vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors. Alternatively, derivatives of viruses such as the bovine papilloma virus (BPV-1), or Epstein-Barr virus (pHEBo, pREP-derived, and p205) can be used for transient expression of proteins in eukaryotic cells. In some embodiments, it may be desirable to express the recombinant protein (e.g., a recombinant glycosylated protein) by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral vectors.

Vectors may be transformed into any suitable mammalian host cell. In some embodiments, the step of transforming the mammalian cell with one or more vectors encoding the one or more sialyltransferases to generate the mammalian host cell includes the performance of supertransfection. Supertransfection involves performing a first transfection of a mammalian cell that uses a first means for integrating (e.g., a first integrating enzyme, e.g., a first integrase, transposon, recombinase, etc.) a first gene into the genome of the mammalian cell and then performing a second transfection of the mammalian cell using a second means for integrating (e.g., same or different as the first means for integrating) (e.g., a second integrating enzyme, e.g., a second integrase, transposon, recombinase, etc.) additional copies of the first gene or a different gene without disruption of the DNA integrated in the first transfection.

In some cells, the vectors are expressed in mammalian host cells to express relatively large quantities of the recombinant glycosylated protein. Such host cells include mammalian cells, yeast cells, insect cells, and prokaryotic cells. In some embodiments, the mammalian host cell can be a Chinese Hamster Ovary (CHO) cell, a baby hamster kidney (BHK) cell, a NS0 cell, a Y0 cell, a HEK293 cell, a COS cell, a Vero cell, or a HeLa cell. In some embodiments, the mammalian host cell is a rodent cell.

The recombinant glycosylated proteins may be secreted and isolated from a mixture of mammalian host cells and liquid culture medium containing the recombinant glycosylated proteins. Alternatively, the recombinant glycosylated proteins may be retained in the cytoplasm or in a membrane fraction and the cells harvested, lysed, and the recombinant glycosylated proteins isolated using a desired method. Exemplary embodiments of the recombinant glycosylated proteins that further include Fc polypeptides are described in WO 2019/070577 (incorporated by reference in its entirety).

### Enzyme Replacement Therapy (ERT) Enzymes

Enzyme replacement therapy (ERT) enzymes are a class of enzymes that are deficient in some subjects. Lysosomal storage disorder (LSD) enzymes is a subclass of ERT enzymes that are deficient in subjects having a LSD. LSDs are inherited metabolic diseases characterized by the accumulation of undigested or partially digested macromolecules, which ultimately results in cellular dysfunction and clinical abnormalities. Classically, LSDs have been defined as deficiencies in lysosomal function generally classified by the accumulated substrate and include sphingolipidoses, oligosaccharidoses, mucolipidoses, mucopolysaccharidoses, lipoprotein storage disorders, neuronal ceroid lipofuscinoses, and others. The classification of these disorders has recently been expanded to include other deficiencies or defects in proteins that result in accumulation of macromolecules, such as proteins necessary for normal post-translational modification of lysosomal enzymes, or proteins important for proper lysosomal trafficking.

In some embodiments, the recombinant glycosylated protein can be an LSD enzyme, or a catalytically active variant or a catalytically active fragment thereof.

In some embodiments, the recombinant glycosylated protein can be an IDUA protein that comprises the amino acid sequence of SEQ ID NO: 3, 4, 8, or 9. In some embodiments, the recombinant glycosylated protein can comprise a catalytically active variant or fragment of an IDUA enzyme has at least 50%, at least 55%, at least 60%, at least 65%, at least 70%, at least 75%, at least 80%, at least 85%, at least 90%, at least 95%, or greater of the activity of the wild-type SGSH enzyme.

In some embodiments, the recombinant glycosylated protein can be an SGSH protein that comprises the amino acid sequence of SEQ ID NO: 1 or 2. In some embodiments, the recombinant glycosylated protein can comprise a catalytically active variant or fragment of an SGSH enzyme has at least 50%, at least 55%, at least 60%, at least 65%, at least 70%, at least 75%, at least 80%, at least 85%, at least 90%, at least 95%, or greater of the activity of the wild-type SGSH enzyme.

In some embodiments, the recombinant glycosylated protein can be an IDS protein that comprises the amino acid sequence of any one of SEQ ID NOS: 5, 6, 7, 12, and 13. In some embodiments, the recombinant glycosylated protein can comprise a catalytically active variant or fragment of an IDS enzyme has at least 50%, at least 55%, at least 60%, at least 65%, at least 70%, at least 75%, at least 80%, at least 85%, at least 90%, at least 95%, or greater of the activity of the wild-type IDS enzyme.

In some embodiments, the recombinant glycosylated protein is a fusion protein (e.g., any of the exemplary fusion proteins described herein or known in the art). In some embodiments, the fusion protein is an antigen binding fusion protein that comprises an antigen binding protein and an ERT enzyme (e.g., an LSD enzyme) that is linked thereto. The antigen binding protein can be, e.g., an antibody or an antigen-binding fragment thereof and encompasses Fab fragments, F(ab′) 2 fragments, Fd fragments, Fv fragments, dAb fragments, scFv fragments, chimeric antibodies, monoclonal antibodies, VNAR domains, and VHH domains.

In some embodiments, the fusion protein comprises: (i) an antibody heavy chain or fragment thereof, and (ii) an antibody light chain or fragment thereof, and an ERT enzyme (e.g., an LSD enzyme) that is linked to (i) and/or (ii). In some embodiments, the ERT enzyme is linked to (i). In some embodiments, the ERT is linked to (ii). In some embodiments, the fusion protein contains at least two ERT enzyme units, wherein the first ERT enzyme unit is linked to (i) and a second ERT enzyme unit is linked to (ii). In some embodiments, the antigen binding fusion protein is capable of specifically binding to a blood-brain barrier (BBB) receptor, e.g. a transferrin receptor (TfR).

In some embodiments, the fusion protein is an ERT enzyme-Fc fusion protein that comprises: (i) an Fc polypeptide, which may contain modifications (e.g., one or more modifications that promote heterodimerization) or may be a wild-type Fc polypeptide; and an ERT enzyme (e.g., an LSD enzyme); and (ii) an Fc polypeptide, which may contain modifications (e.g., one or more modifications that promote heterodimerization) or may be a wild-type Fc polypeptide; and an ERT enzyme (e.g., an LSD enzyme). In some embodiments, the fusion protein comprises (i) a first Fc polypeptide that is linked to an enzyme replacement therapy (ERT) enzyme, a catalytically active ERT enzyme variant, or a catalytically active ERT enzyme fragment; and (ii) a second Fc polypeptide that forms an Fc dimer with the first Fc polypeptide, wherein the first Fc polypeptide and/or the second Fc polypeptide is a modified Fc polypeptide capable of specifically binding to a blood-brain barrier (BBB) receptor, e.g. a transferrin receptor (TfR). In other embodiments, the fusion protein comprises (i) an enzyme replacement therapy (ERT) enzyme, a catalytically active ERT enzyme variant, or a catalytically active ERT enzyme fragment thereof; and (ii) a modified Fc dimer that is capable of specifically binding to a transferrin receptor (TfR). In some embodiments, one or both Fc polypeptides may contain modifications that result in binding to a blood-brain barrier (BBB) receptor, e.g., a transferrin receptor (TfR).

In some embodiments, the ERT enzyme can be an LSD enzyme. An LSD enzyme incorporated into the fusion protein is catalytically active, i.e., it retains the enzymatic activity that is deficient in the LSD. In some embodiments, the LSD enzyme is alpha-L-iduronidase (IDUA), which is deficient in Sanfilippo syndrome. In some embodiments, the LSD enzyme is alpha-L-iduronidase (IDUA), which is deficient in mucopolysaccharidosis type I. In some embodiments, the LSD enzyme is iduronate 2-sulfatase (IDS), which is deficient in Hunter syndrome. In some embodiments, the LSD enzyme is acid sphingomyelinase (ASM), which is deficient in Niemann-Pick disease. In some embodiments, the LSD enzyme is β-glucocerebrosidase (GBA), which is deficient in Gaucher's disease and Parkinson's disease.

In some embodiments, a fusion protein comprising an LSD enzyme and optionally a modified Fc polypeptide that binds to a BBB receptor, e.g., a TfR-binding Fc polypeptide, comprises SGSH or a catalytically active fragment or variant of a wild-type SGSH. In some embodiments, the SGSH is a catalytically active variant or a catalytically active fragment of an SGSH protein that comprises the amino acid sequence of any one of SEQ ID NOS: 1 and 2. In some embodiments, a catalytically active variant or fragment of a SGSH protein has at least 50%, at least 55%, at least 60%, at least 65%, at least 70%, at least 75%, at least 80%, at least 85%, at least 90%, at least 95%, or greater of the activity of the wild-type SGSH protein. In some embodiments, a fusion protein comprising SGSH and a modified Fc polypeptide can comprise a first polypeptide comprising a sequence that is at least 80%, at least 85%, at least 90%, at least 95%, at least 99%, or 100% identical to SEQ ID NO: 14, 15, 16, 17, or 18, and a second polypeptide comprising a sequence that is at least 80%, at least 85%, at least 90%, at least 95%, at least 99%, or 100% identical to SEQ ID NO: 18, 19, 20, or 21.

In some embodiments, a fusion protein comprising an LSD enzyme and optionally a modified Fc polypeptide that binds to a BBB receptor, e.g., a TfR-binding Fc polypeptide, comprises IDUA or a catalytically active fragment or variant of a wild-type IDUA. In some embodiments, the IDUA is a catalytically active variant or a catalytically active fragment of an IDUA protein that comprises the amino acid sequence of any one of SEQ ID NOS: 3, 4, 8 and 9. In some embodiments, a catalytically active variant or fragment of IDUA protein has at least 50%, at least 55%, at least 60%, at least 65%, at least 70%, at least 75%, at least 80%, at least 85%, at least 90%, at least 95%, or greater of the activity of the wild-type IDUA protein.

In some embodiments, a fusion protein comprising IDUA and a modified Fc polypeptide can comprise a first polypeptide comprising a sequence that is at least 80%, at least 85%, at least 90%, at least 95%, at least 99%, or 100% identical to SEQ ID NO: 22 or 23 and a second polypeptide comprising a sequence that is at least 80%, at least 85%, at least 90%, at least 95%, at least 99%, or 100% identical to SEQ ID NO: 24 or 25.

In some embodiments, a fusion protein comprising an ERT enzyme and optionally a modified Fc polypeptide that binds to a BBB receptor, e.g., a TfR-binding Fc polypeptide, comprises a IDS or a catalytically active fragment or variant of a wild-type IDS. In some embodiments, the IDS is a catalytically active variant or a catalytically active fragment of an IDS protein that comprises the amino acid sequence of any one of SEQ ID NOS: 5, 6, 7, 12, and 13. In some embodiments, a catalytically active variant or fragment of an IDS protein has at least 50%, at least 55%, at least 60%, at least 65%, at least 70%, at least 75%, at least 80%, at least 85%, at least 90%, at least 95%, or greater of the activity of the wild-type IDS protein.

In some embodiments, an ERT enzyme, e.g., IDUA, SGSH, or IDS, or a catalytically active variant or fragment thereof, that is present in a fusion protein described herein, retains at least 25% of its activity compared to its activity when not joined to an Fc polypeptide or a TfR-binding Fc polypeptide. In some embodiments, an ERT enzyme, or a catalytically active variant or a catalytically active fragment thereof, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, an ERT enzyme, or a catalytically active ERT enzyme variant or a catalytically active ERT enzyme fragment, retains at least 80%, 85%, 90%, or 95% of its activity compared to its activity when not joined to an Fc polypeptide or a TfR-binding Fc polypeptide. In some embodiments, fusion to an Fc polypeptide does not decrease the activity of the ERT enzyme, e.g., IDUA, SGSH, or IDS, or catalytically active variant or fragment thereof. In some embodiments, fusion to a TfR-binding Fc polypeptide does not decrease the activity of the ERT enzyme.

### Transferrin Receptor-Binding Fc Polypeptides

This section describes generation of modified Fc polypeptides described herein that bind to transferrin receptor (TfR) and are capable of being transported across the blood-brain barrier (BBB).

### TfR-Binding Fc Polypeptides Comprising Mutations in the CH3 Domain

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 384, 386, 387, 388, 389, 390, 413, 416, and 421, according to the EU numbering scheme. Illustrative substitutions that may be introduced at these positions are shown in Tables 4 and 5. In some embodiments, the amino acid at position 388 and/or 421 is an aromatic amino acid, e.g., Trp, Phe, or Tyr. In some embodiments, the amino acid at position 388 is Trp. In some embodiments, the aromatic amino acid at position 421 is Trp or Phe.

In some embodiments, at least one position as follows is substituted: Leu, Tyr, Met, or Val at position 384; Leu, Thr, His, or Pro at position 386; Val, Pro, or an acidic amino acid at position 387; an aromatic amino acid, e.g., Trp at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, an Fc polypeptide that is modified as described in the preceding two paragraphs comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415, according to the EU numbering scheme. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413: Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified Fc polypeptide further comprises Ser, Thr, Gln, or Phe at position 391. In some embodiments, the modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, the modified Fc polypeptide does not have a Trp at position 380.

In other embodiments, the modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Trp at position 380 and/or Glu at position 415.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide comprises one or more of the following substitutions: Trp at position 380; Thr at position 386; Trp at position 388; Val at position 389; Thr or Ser at position 413; Glu at position 415; and/or Phe at position 421, according to the EU numbering scheme.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOS: 4-90, 97-100, and 105-108 (e.g., SEQ ID NOS: 34-38, 58, and 60-90) as described in WO 2019/070577, incorporated by reference in its entirety. In some embodiments, the modified Fc polypeptide has at least 70% identity, at least 75% identity, at least 80% identity, at least 85% identity, at least 90% identity, or at least 95% identity to any one of SEQ ID NOS: 4-90, 97-100, and 105-108 (e.g., SEQ ID NOS: 34-38, 58, and 60-90) as described in WO 2019/070577, incorporated by reference in its entirety. In some embodiments, the modified Fc polypeptide comprises the amino acids at EU index positions 384-390 and/or 413-421 of any one of SEQ ID NOS: 4-90, 97-100, and 105-108 (e.g., SEQ ID NOS: 34-38, 58, and 60-90) as described in WO 2019/070577, incorporated by reference in its entirety. In some embodiments, the modified Fc polypeptide comprises the amino acids at EU index positions 380-390 and/or 413-421 of any one of SEQ ID NOS: 4-90, 97-100, and 105-108 (e.g., SEQ ID NOS: 34-38, 58, and 60-90) as described in WO 2019/070577, incorporated by reference in its entirety. In some embodiments, the modified Fc polypeptide comprises the amino acids at EU index positions 380-392 and/or 413-426 of any one of SEQ ID NOS: 4-90, 97-100, and 105-108 (e.g., SEQ ID NOS: 34-38, 58, and 60-90) as described in WO 2019/070577, incorporated by reference in its entirety.

In some embodiments, the modified Fc polypeptide has at least 75% identity, at least 80% identity, at least 85% identity, at least 90% identity, or at least 95% identity to any one of SEQ ID NOS: 4-90, 97-100, and 105-108 (e.g., SEQ ID NOS: 34-38, 58, and 60-90), as described in WO 2019/070577, incorporated by reference in its entirety, and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOS: 34-38, 58, and 60-90, as described in WO 2019/070577, incorporated by reference in its entirety. In other embodiments, the modified Fc polypeptide comprises the amino acid sequence of any one of SEQ ID NOS: 34-38, 58, and 60-90, as described in WO 2019/070577, incorporated by reference in its entirety, but in which one, two, or three amino acids are substituted.

In some embodiments, the modified Fc polypeptide comprises additional mutations such as the mutations described in Section VI below, including, but not limited to, a knob mutation (e.g., T366W as numbered with reference to EU numbering), hole mutations (e.g., T366S, L368A, and Y407V as numbered with reference to EU numbering), mutations that modulate effector function (e.g., L234A and L235A; L234A, L235A and P329G; or L234A, L235A, and P329S) as numbered with reference to EU numbering), and/or mutations that increase serum stability or serum half-life (e.g., (i) M252Y, S254T, and T256E as numbered with reference to EU numbering, or (ii) N434S with or without M428L as numbered according to the EU numbering scheme). By way of illustration, SEQ ID NOS: 156-229 provide non-limiting examples of modified Fc polypeptides with mutations in the CH3 domain (e.g., clones CH3C.35.20.1, CH3C.35.23.2, CH3C.35.23.3, CH₃C.35.23.4, CH3C.35.21.17.2, and CH3C.35.23) comprising one or more of these additional mutations.

In some embodiments, the modified Fc polypeptide comprises a knob mutation (e.g., T366W as numbered with reference to EU numbering) and has at least 85% identity, at least 90% identity, or at least 95% identity to the sequence of any one of SEQ ID NOS: 156, 168, 180, 192, 204, and 216, as described in WO 2019/070577, incorporated by reference in its entirety. In some embodiments, the modified Fc polypeptide comprises the sequence of any one of SEQ ID NOS: 156, 168, 180, 192, 204, and 216, as described in WO 2019/070577, incorporated by reference in its entirety.

In some embodiments, the modified Fc polypeptide comprises a knob mutation (e.g., T366W as numbered with reference to EU numbering) and mutations that modulate effector function (e.g., L234A and L235A, L234A, L235A, and P329G; or L234A, L235A, and P329S) as numbered with reference to EU numbering), and has at least 85% identity, at least 90% identity, or at least 95% identity to the sequence of any one of SEQ ID NOS: 157, 158, 169, 170, 181, 182, 193, 194, 205, 206, 217, 218, 228, and 229, as described in WO 2019/070577, incorporated by reference in its entirety. In some embodiments, the modified Fc polypeptide comprises the sequence of any one of SEQ ID NOS: 157, 158, 169, 170, 181, 182, 193, 194, 205, 206, 217, and 218, as described in WO 2019/070577, incorporated by reference in its entirety.

In some embodiments, the modified Fc polypeptide comprises hole mutations (e.g., T366S, L368A, and Y407V as numbered with reference to EU numbering) and has at least 85% identity, at least 90% identity, or at least 95% identity to the sequence of any one of SEQ ID NOS: 162, 174, 186, 198, 210, and 222, as described in WO 2019/070577, incorporated by reference in its entirety. In some embodiments, the modified Fc polypeptide comprises the sequence of any one of SEQ ID NOS: 162, 174, 186, 198, 210, and 222, as described in WO 2019/070577, incorporated by reference in its entirety.

In some embodiments, the modified Fc polypeptide comprises hole mutations (e.g., T366S, L368A, and Y407V as numbered with reference to EU numbering) and mutations that modulate effector function (e.g., L234A and L235A; L234A, L235A, and P329G; or L234S, L235A, and P329S) as numbered with reference to EU numbering), and has at least 85% identity, at least 90% identity, or at least 95% identity to the sequence of any one of SEQ ID NOS: 163, 164, 175, 176, 187, 188, 199, 200, 211, 212, 223, and 224, as described in WO 2019/070577, incorporated by reference in its entirety. In some embodiments, the modified Fc polypeptide comprises the sequence of any one of SEQ ID NOS: 163, 164, 175, 176, 187, 188, 199, 200, 211, 212, 223, and 224, as described in WO 2019/070577, incorporated by reference in its entirety.

### Sialyltransferases

Sialyltransferases are enzymes that transfer sialic acid to oligosaccharides. Each sialyltransferase is specific for a particular substrate. Sialyltransferases add sialic acid to the terminal portions of the sialylated glycolipids or to the N- or O-linked sugar chains of glycoproteins. Sialyltransferases belong to glycosyltransferase family 29. There are twenty different sialyltransferases.

In some embodiments, the mammalian host cell has been previously transformed with a vector encoding one or more sialyltransferases (e.g., ST6GAL1 and/or ST3GAL4).

“ST6GAL1” as used herein refers to beta-galactoside alpha-2,6-sialyltransferase 1, which transfers sialic acid from CMP-sialic acid to galactose-containing acceptor substrates. ST6GAL1 protein is normally found within the Golgi but can be proteolytically processed to a soluble form. The sequence of human ST6GAL11, which is the human sequence designated as the canonical sequence, is available under UniProt entry P15907 and is encoded by the human ST6GAL1 gene at 3q27.3. The full-length sequence is provided as SEQ ID NO: 18. There are 13 potential isoforms of ST6GAL1. Exemplary isoforms of ST6GAL1 available are under UniProt entry C9J6X5, C9KOR8, C9JH16, and H7C472. An exemplary ST6GAL1 gene is available under NBCI Accession No. NP_001340845.1.

In some embodiments, the mammalian host cell has been previously transformed with a vector encoding ST6GAL1. In some embodiments, the ST6GAL1 can have the amino acid sequence of any one of SEQ ID NOS: 26, 27, 28, 29, and 30, or a catalytically active variant or catalytically active fragment thereof. In some embodiments, a catalytically active variant or fragment of an ST6GAL1 protein has at least 50%, at least 55%, at least 60%, at least 65%, at least 70%, at least 75%, at least 80%, at least 85%, at least 90%, at least 95%, or greater of the activity of the wild-type ST6GAL1 protein. In some embodiments, the ST6GAL1 can comprise a sequence that is at least 80% identical, at least 85% identical, at least 90% identical, at least 95% identical, at least 99% identical, or 100% identical to any one of SEQ ID NOS: 26, 27, 28, 29, and 30.

“ST3GAL4” as used herein refers to CMP-N-acetylneuraminate-beta-galactosamide-alpha-2,3-siailtransferase 4, which catalyzes the transfer of sialic acid from CMP-Neu5 Ac onto acceptor Galbeta-(1->3)-GalNAc- and Galbeta-(1->4)-GlcNAc-terminated glycoconjugates through an alpha2-3 linkage. ST3GAL4 protein is normally found within the Golgi but can be proteolytically processed and secreted. The sequence of human ST3GAL4, which is the human sequence designated as the canonical sequence, is available under UniProt entry Q11206 and is encoded by the human ST3GAL4 gene at 11q24.2. The full-length sequence is provided as SEQ ID NO: 23. Exemplary ST3GAL4 genes are available under NBCI Accession No. XP_047283377.1 or NCBI Accession No. NP_001233628.1.

In some embodiments, the mammalian host cell has been previously transformed with a vector encoding ST3GAL4. In some embodiments, the ST3GAL4 can have the amino acid sequence of any one of SEQ ID NOS: 30, 31, 32, 33, and 34, or a catalytically active variant or catalytically active fragment thereof. In some embodiments, a catalytically active variant or fragment of an ST3GAL4 protein has at least 50%, at least 55%, at least 60%, at least 65%, at least 70%, at least 75%, at least 80%, at least 85%, at least 90%, at least 95%, or greater of the activity of the wild-type ST3GAL4 protein. In some embodiments, the ST3GAL4 can comprise a sequence that is at least 80% identical, at least 85% identical, at least 90% identical, at least 95% identical, at least 99% identical, or 100% identical to any one of SEQ ID NOS: 30, 31, 32, 33, and 34.

### Media Additives

Liquid culture media (culture media) are known in the art. A liquid culture media can comprise one or more media additive(s). In some embodiments, the liquid culture media comprises one or more (e.g., 1, 2, 3, or 4) media additives selected from the group of hydrocortisone, N-acetylmannosamine (ManNAc), manganese, and uridine. In some embodiments, the liquid culture media comprises at least one (e.g., at least two, at least three) media additives selected from the group consisting of: hydrocortisone, N-acetylmannosamine (ManNAc), manganese, and uridine. In some embodiments, the liquid culture media comprises hydrocortisone, N-acetylmannosamine (ManNAc), manganese, and uridine.

In some embodiments of any of the methods described herein, the ManNAc is present in the liquid culture medium at a concentration of from about 10 mM to about 60 mM (e.g., about 10 mM to about 50 mM, about 10 mM to about 40 mM, or about 10 mM to about 30 mM).

In some embodiments of any of the methods described herein, the uridine is present in the liquid culture medium at a concentration of from about 2.5 mM to about 10 mM (e.g., about 2.5 mM to about 8 mM, about 2.5 mM to about 6 mM, or about 2.5 mM to about 5 mM).

In some embodiments of any of the methods described herein, the hydrocortisone is present in the liquid culture medium at a concentration of from about 5 μM to about 50 μM (e.g., about 5 μM to about 40 μM, 5 μM to about 30 μM, or 5 μM to about 20 μM).

In some embodiments of any of the methods described herein, the manganese is present in the liquid culture medium at a concentration of from about 2 μM to about 15 μM (e.g., about 2 μM to about 10 UM or about 2 μM to about 5 μM).

In some embodiments of any of the methods described herein, the liquid culture medium comprises about 10 mM to about 60 mM (or any of the exemplary subranges described herein) ManNAc and about 2.5 mM to about 10 mM (or any of the exemplary subranges described herein) uridine. In some embodiments of any of the methods described herein, the liquid culture medium comprises about 10 mM ManNAc and about 2.5 mM uridine. In some embodiments of any of the methods described herein, the liquid culture medium comprises about 10 mM ManNAc and about 5 mM uridine.

In some embodiments of any of the methods described herein, the liquid culture medium comprises about 10 mM to about 60 mM (or any of the exemplary subranges described herein) ManNAc and about 2 μM to about 15 μM (or any of the exemplary subranges described herein) manganese. In some embodiments of any of the methods described herein, the liquid culture medium comprises about 10 mM ManNAc and about 10 μM manganese.

In some embodiments of any of the methods described herein, the liquid culture medium comprises about 2.5 mM to about 10 mM (or any of the exemplary subranges described herein) uridine and about 2 μM to about 15 μM (or any of the exemplary subranges described herein) manganese. In some embodiments of any of the methods described herein, the liquid culture medium comprises about 2.5 mM uridine and about 10 μM manganese. In some embodiments of any of the methods described herein, the liquid culture medium comprises about 5 mM uridine and about 10 μM manganese.

In some embodiments of any of the methods described herein, the liquid culture medium comprises about 10 mM to about 60 mM (or any of the exemplary subranges described herein) ManNAc, about 2.5 mM to about 10 mM (or any of the exemplary subranges described herein) uridine, and about 2 μM to about 15 μM (or any of the exemplary subranges described herein) manganese. In some embodiments of any of the methods described herein, the liquid culture medium comprises about 10 mM ManNAc, about 2.5 mM uridine, and about 10 μM manganese. In some embodiments of any of the methods described herein, the liquid culture medium comprises about 10 mM ManNAc, about 20 μM hydrocortisone, about 2.5 mM uridine, and about 10 UM manganese.

In some embodiments of any of the methods described herein, the liquid culture medium comprises about 10 mM to about 60 mM (or any of the exemplary subranges described herein) ManNAc, about 2.5 mM to about 10 mM (or any of the exemplary subranges described herein) uridine, about 5 μM to about 50 μM (or any of the exemplary subranges described herein) hydrocortisone, and about 2 μM to about 15 μM (or any of the exemplary subranges described herein) manganese.

The liquid culture medium used in any of the steps of any of the methods described herein can be any of the types of liquid culture medium known in the art.

### Types of Culturing

The term “batch culturing” is a term of art and means the culturing a cell culture in a vessel (e.g., a bioreactor), wherein culturing the cell culture in the vessel does not include the addition and/or the removal of a substantial amount of liquid culture medium present in the vessel (e.g., liquid culture medium that is at least 90% free of host cells) during the cell culturing period. Batch culturing can be performed using temperatures, CO₂ gas exposures and/or agitation rates known to those in the art. One skilled in the art would appreciate that the length of time of batch culturing the cell culture will depend on the growth rate of the host cells, the recombinant glycosylated protein, and the starting cell culture density.

The term “fed-batch culturing” is a term of art and means the culturing a cell culture in a vessel (e.g., a bioreactor), wherein culturing the cell culture in the vessel includes the periodic or continuous addition of fresh liquid culture medium and/or growth feeds (e.g., glucose) to the vessel without the removal of a substantial amount of liquid culture medium present in the vessel. The fresh liquid culture medium can be the same liquid culture medium as the liquid culture medium present in the vessel at the start of the culturing period. In some examples of fed-batch culturing, the fresh liquid culture medium is a different liquid culture medium and/or includes one or more different media additives (e.g., any of the media additives described herein). Fed-batch culturing can be performed using temperatures, CO₂ gas exposures and/or agitation rates known to those in the art. One skilled in the art would appreciate that the length of time of fed-batch culturing the cell culture will depend on the growth rate of the host cells, the recombinant glycosylated protein, and the starting cell culture density.

The term “perfusion culturing” is a term of art and means the culturing of a cell culture in a vessel (e.g., a bioreactor), wherein culturing the cell culture in the vessel includes the periodic or continuous removal of liquid culture medium present in the vessel (e.g., liquid culture medium that is at least 90% free of host cells) during the cell culture period, and simultaneously or shortly thereafter adding substantially the same volume of a replacement liquid culture medium or a fresh liquid culture medium to the vessel. The replacement liquid culture medium or fresh liquid culture medium can be the same liquid culture medium as the liquid culture medium present in the vessel at the start of the culturing period. In some examples of perfusion culturing, the replacement liquid culture medium or fresh liquid culture medium is a different liquid culture medium and/or includes one or more different media additives (e.g., any of the media additives described herein). Perfusion culturing can be performed using temperatures, CO₂ gas exposures and/or agitation rates known to those in the art. One skilled in the art would appreciate that the length of time of perfusion culturing the cell culture will depend on the growth rate of the host cells, the recombinant glycosylated protein, and the starting cell culture density.

Methods of Determining the Level of N-Acetylneuraminic acid (NeuAc or Sialic Acid) in a Recombinant Glycosylated Protein

Methods for determining the level of sialic acid in a recombinant glycosylated protein are known in the art. In some embodiments, the methods provided herein result in an increase in the sialic acid level in the recombinant glycosylated protein by at least 50% (e.g., at least 75%, at least 95%, at least 100%, at least 150%, at least 200, at least 250%, 50%, 75%, 95%, 100%, 150%, 200%, or 250%) as compared to the recombinant glycosylated protein produced by a method that does not include one or both of (i) the use of a mammalian host cell (e.g., any of the mammalian cells described herein) capable of expressing the recombinant glycosylated protein comprising one or more vectors encoding the one or more (e.g., 1, 2, 3, or 4) sialyltransferases (e.g., any of the sialyltransferases described herein), and (ii) culturing a mammalian host cell (e.g., any of the mammalian cells described herein) capable of expressing the recombinant glycosylated protein in a liquid culture medium comprising one or more (e.g., 1, 2, 3, or 4) media additives selected from the group consisting of: hydrocortisone, N-acetylmannosamine (ManNAc), manganese, and uridine.

In an exemplary embodiment, NeuAc (sialic acid) content in a recombinant glycosylated protein (e.g., a recombinant glycosylated protein produced using any of the methods described herein) can be measured by liquid chromatography-mass spectrometry analysis or by reversed-phase liquid chromatography analysis. Embodiments for preparation of samples for measuring NeuAc content are described in the Examples disclosed herein.

### Methods of Harvesting and Isolating Recombinant Glycosylated Protein

In some embodiments of any of the methods described herein, the method further includes harvesting the recombinant glycosylated protein from the liquid culture medium and/or the mammalian host cell (e.g., any of the mammalian host cells described herein). In some embodiments of any of the methods described herein, the method further includes isolating the harvested recombinant glycosylated protein.

A recombinant glycosylated protein can be recovered from the liquid culture medium by removing or otherwise physically separating the liquid culture medium from the mammalian host cell (e.g., any of the mammalian host cells described herein). A variety of different methods for removing liquid culture medium from mammalian host cells are known in the art, including, for example, centrifugation, filtration, pipetting, and/or aspiration. The recombinant glycosylated protein can then be recovered and isolated from the liquid culture medium using a variety of biochemical techniques, including affinity chromatography, hydrophobic interaction chromatography, and/or size exclusion chromatography.

### Methods of Formulating an Isolated Recombinant Glycosylated Protein

In some embodiments of any of the methods described herein, the method further includes formulating the isolated recombinant glycosylated protein. Any of the isolated recombinant glycosylated proteins can be formulated for parenteral (e.g., intravenous, intramuscular, intradermal, subcutaneous, intraarterial, or intraperitoneal) administration in dosage unit form (i.e., physically discrete units containing a predetermined quantity of active recombinant glycosylated protein for ease of administration and uniformity of dosage). Data obtained from cell culture assays and animal studies can be used in formulating an appropriate dosage of any given isolated recombinant glycosylated protein for use in a subject (e.g., a human).

### Methods of Treatment

Provided herein are recombinant glycosylated proteins (e.g., isolated recombinant glycosylated proteins) produced by any of the methods described herein. Also provided herein are methods of treating a subject in need thereof that include administering to the subject a therapeutically effective amount of any of the recombinant glycosylated proteins produced using the methods described herein. For example, a recombinant glycosylated protein that comprises a LSD enzyme or a variant or catalytically active fragment thereof can be used to treat an LSD. In some embodiments, a patient having Hunter syndrome is treated with a recombinant glycosylated protein that comprises IDS or a variant or catalytically active fragment thereof. In some embodiments, a patient having mucopolysaccharidosis type I is treated with a recombinant glycosylated protein comprising IDUA or a variant or a catalytically active fragment thereof. In some embodiments, a patient having Sanfilippo syndrome A is treated with a recombinant glycosylated protein comprising SGSH or a variant or catalytically active fragment thereof.

Any of the recombinant glycosylated proteins produced using any of the methods described herein can be administered to a subject in need thereof at a therapeutically effective amount or dose. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient.

In various embodiments, a recombinant glycosylated protein produced by the methods described herein is administered parenterally. In some embodiments, the recombinant glycosylated protein is administered intravenously.

### EXAMPLES

The invention is further described in the following examples, which do not limit the scope of the invention described in the claims.

### Example 1. Effect of Media Additives on Expression of an Enzyme Fusion Protein

An exemplary enzyme fusion protein containing SGSH enzyme (“SGSH-Fc fusion protein”) was used to test the effect of different media additives on sialic acid levels produced by clonal cells expressing the fusion protein.

Sialic Acid Levels were Improved by Addition of Media Additives

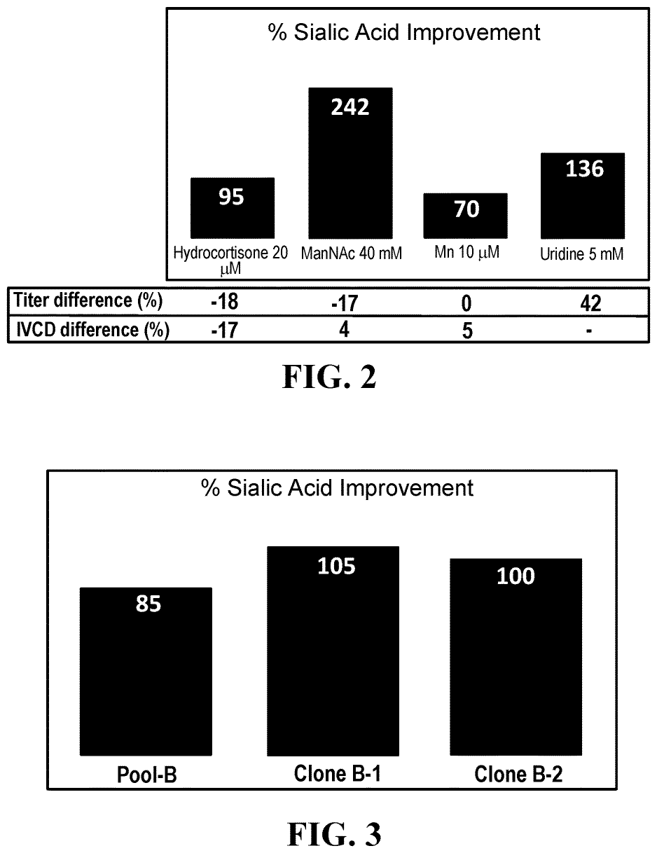

Different media additives at varying concentrations were tested on clones expressing SGSH-Fc fusion protein to determine if sialic acid levels and other product quality attributes could be improved relative to that produced in the absence of the media additives. As illustrated in FIG. 2 and Table 1, each of hydrocortisone, N-acetylmannosamine (ManNAc), manganese (Mn), and uridine resulted in an increase in sialic acid levels in the fusion protein relative to that produced in the absence of the additives to the media. For example, the following additives each resulted in at least a 70% increase in sialic acid levels in the fusion protein: (1) hydrocortisone, 20 μM; (2) ManNAc, 40 μM; (3) Mn, 10 μM; and (4) uridine, 5 mM. The presence of uridine also contributed to an increase in protein titer (about 42% increase).

TABLE 1
Effect of media additives on sialic acid level
content and titer in SGSH-Fc fusion proteins

| | Sialic | Sialic Acid | Titer |
|---|---|---|---|
| | Acid | Improvement | Difference |
| Condition | (mol/mol) | (%) | (%) |
| Hydrocortisone 0 uM | 1.29 | 0.0 | 0.0 |
| Hydrocortisone 5 uM | 2.16 | 67.4 | 0.3 |
| Hydrocortisone 10 uM | 2.24 | 73.6 | −6.7 |
| Hydrocortisone 20 uM | 2.52 | 95.3 | −17.9 |
| ManNAc 0 mM | 0.56 | 0.0 | 0.0 |
| ManNAc 0 mM | 0.67 | | |
| ManNAc 10 mM | 1.25 | 107.3 | −6.9 |
| ManNAc 10 mM | 1.3 | | |
| ManNAc 20 mM | 1.62 | 156.1 | −13.8 |
| ManNAc 20 mM | 1.53 | | |
| ManNAc 40 mM | 2.23 | 242.3 | −17.2 |
| ManNAc 40 mM | 1.98 | | |
| Mn 10 uM | 1.01 | 69.9 | 0.0 |
| Mn 10 uM | 1.08 | | |
| Uridine 0 mM | 0.77 | 0.0 | 0.0 |
| Uridine 5 mM | 1.82 | 136.4 | 42.6 |

### Recombinant Protein Expression and Purification

Stable CHO pool was generated by co-transfection of transposons coding for SGSH-Fc fusion protein and hSUMF1 (NCBI Accession No. NP_877437.2) at mass ratio of 4:1 and transposase mRNA into GS-KO CHO-K1 cells (Horizon) by electroporation and subsequent selection in a commercially available, chemically-defined, animal component-free CHO growth medium that is glutamine free. Subsequently, the stable pool was subjected to single cell cloning followed by clone screening to identify a clone with desirable titer and product quality attributes. Effect of media additives was evaluated in a 14-day fed-batch production process with the clone. On day 0, cells were seeded at about 0.5×10⁶ cells/mL density in production medium. The cultures were maintained at 37° C. in 5% CO₂ and 85% relative humidity. When cultures reached a density of about 10-15×10⁶ cells/mL, they were shifted to 32° C. for the remaining duration of the run. Commercially available feeds were added on days 3, 5, 7, 10 and 12. Media additives were added in 2-3 aliquots distributed over the entire run time. Cultures were harvested on day 14 by centrifugation (5000×g, 15-20 min) followed by sterile filtration through a 0.22 μm filter and stored at 4° C.

SGSH-Fc fusion proteins were purified from cell culture supernatants using Protein A affinity chromatography. Supernatants were loaded onto an equilibrated (Tris acetate, NaCl pH 7.4) Protein A affinity drip column which was then washed with a high conductivity Tris acetate, NaCl pH 7.4 buffer. After re-equilibration of the column, the bound proteins were then eluted using a low pH sodium acetate buffer. The eluted proteins were then neutralized using a Tris pH 8 buffer to a final pH of 5.0. Homogeneity of SGSH-Fc fusions in eluted fractions was assessed by a number of techniques including reducing and non-reducing SDS-PAGE and HPLC-SEC.

The SGSH-Fc fusion proteins that were expressed and purified comprise a first SGSH-Fc fusion polypeptide having the sequence of any one of SEQ ID NOS: 14 and 16 and a second SGSH-Fc fusion polypeptide that binds TfR having the sequence of any one of SEQ ID NOS: 19 and 20. During cell culture production, the SGSH-Fc fusion protein may also be further processed such that the first SGSH-Fc fusion polypeptide has the sequence of SEQ ID NO: 15 or 17 and/or the second SGSH-Fc fusion polypeptide that binds TfR has the sequence of SEQ ID NO: 19 or 21. Thus, the fusion proteins may contain unprocessed sequences i.e., SEQ ID NOs: 14, 16, 18, and 20); one or more processed sequences (i.e., selected from SEQ ID NOs: 15, 17, 19, and 21); or a mixture comprising processed and unprocessed protein molecules.

### Measurement of N-Acetylneuraminic Acid (NeuAc or Sialic Acid) Content

NeuAc (sialic acid) content in the SGSH-Fc fusion proteins were measured one of two methods described below.

In the first method, NeuAc (sialic acid) content was measured by liquid chromatography-mass spectrometry analysis. Five (5) μg of protein in PBS (1×, pH 7.4) was transferred into each well of a 96-well microplate. Two hundred (200) ng of internal standard (1,2,3-¹³C NeuAc, Omicronbio Inc, Cat #N-acetyl-D-[1,2,3-¹³C3] neuraminic acid (NEU-004)) was spiked into each well. Samples were brought to a final volume of 30 μL by addition of 1 M Tris HCl buffer (final concentration at 0.1 M) and ultrapure water. Two (2) μL of SialEXO (10 units/μL) (Genovis Inc. PN: G1-SM1-020) were added to each sample. The plate was then sealed and incubated 37° C. for 2 hours with shaking. After the incubation period, the plates were centrifuged, and 2 μL of sample was directly injected onto an LC column. NeuAc analyses were performed by liquid chromatography on UHPLC Vanquish (Thermo Scientific, CA, USA) coupled to UV/Vis and Q Exactive Orbitrap electrospray ionization mass spectrometer (Thermo Scientific, CA, USA) as described above for M6P analysis. Data was collected using parallel reaction monitoring (PRM) acquisition under negative mode including NeuAc and NeuAc internal standard (IS), inclusion time 1.0 to 1.8 min, with precursors at 308.0992 (NeuAc) and 311.1175 (NeuAc-IS). AUC ratios of NeuAc/NeuAc-IS were used to calculate the molecular amount of NeuAc released from protein and the mol of NeuAc per mol of protein was obtained.

In the second method, the sialic acid content was determined using a reversed-phase liquid chromatography method that measures the amount of N-acetylneuraminic acid (Neu5Ac) and N-glycolylneuraminic acid (Neu5Gc). SGSH-Fc test samples were desalted, and sialic acid was released by acid hydrolysis using acetic acid. Released sialic acid was labelled with 4,5-Methylenedioxy-1,2-phenylenediamine dihydrochloride (DMB). Standard solutions of Neu5 Ac and Neu5Gc were also labeled using DMB and were used for generating calibration curves. The DMB-labeled Neu5Ac and Neu5Gc were separated on a reverse phase C-18 HPLC column using a gradient elution and monitored using a fluorescence detector (FLD). The sialic acid content of the test sample was quantified by interpolation of the calibration curve and reported as a molar ratio (mol sialic acid per mol protein).

### Example 2. Effect of a Media Additive Combination on the Production of an Enzyme Fusion Protein

An exemplary enzyme fusion protein containing IDUA enzyme (“IDUA-Fc fusion protein”) was used to test the effect of a combination of media additives on sialic acid levels produced by clonal cells expressing the fusion protein.

Sialic Acid Levels were Improved by a Combination of Media Additives

A media additive combination (10 mM ManNAc, 10 μM Mn, and 2.5 mM uridine) was tested on clones expressing IDUA-Fc fusion proteins to determine if sialic acid levels and other product quality attributes could be improved relative to that produced in the absence of the combination. As illustrated in FIG. 3 and Table 2, the combination of media additives resulted in at least an 80% increase in sialic acid levels in the fusion proteins relative to that produced in the absence of the combination.

TABLE 2
Effect of the media additive combination on sialic acid level
content and product quality in IDUA-Fc fusion proteins

| | | Sialic Acid | |
|---|---|---|---|
| Cell line | Condition | (mol/mol) | SA Improvement (%) |
| Pool B | Control | 2.0 | |
| | Combination | 3.6 | 84.6 |
| Clone B-1 | Control | 1.9 | |
| | Combination | 3.9 | 105.3 |
| Clone B-2 | Control | 1.6 | |
| | Combination | 3.2 | 100.0 |

Control = no additives
Combination = 10 mM ManNAc, 10 μM Mn, and 2.5 mM uridine

### Recombinant Protein Expression and Purification

Stable CHO pool was generated by transfection of transposons coding for IDUA-Fc fusion protein and transposase mRNA into GS-KO CHO-K1 cells (Horizon) by electroporation and subsequent selection in a commercially available, chemically-defined, animal component-free CHO growth medium that is glutamine-free. Subsequently, the stable pool was subjected to single cell cloning followed by clone screening to identify clones (e.g., Clones B-1, B-2) with desirable titer and product quality attributes. Effect of media additives was evaluated in a 14-day fed-batch production process with either pool or clones. On day 0, cells were seeded at 0.5×10⁶ cells/mL density in production medium. The cultures were maintained at 37° C. in 5% CO₂ and 85% relative humidity. When cultures reached a density of 10-15×10⁶ cells/mL, they were shifted to 32° C. for the remaining duration of the run. Commercially available feeds were added on days 3, 5, 7, 10 and 12. Media additives were added in 2-3 aliquots distributed over the entire run time. Cultures were harvested on day 14 by centrifugation (5000×g, 15-20 min) followed by sterile filtration through a 0.22 μm filter and stored at 4° C.

IDUA-Fc fusion proteins were purified from cell culture supernatants using Protein A affinity chromatography. Supernatants were loaded onto an equilibrated (PBS) Protein A affinity drip column which was then re-equilibrated before the bound proteins were eluted using a low pH sodium acetate buffer. The eluted proteins were then neutralized using a Tris pH 8 buffer to a final pH of 5.5.

The IDUA-Fc fusion proteins that were expressed and purified comprise a TfR-binding modified Fc polypeptide having the sequence of SEQ ID NO: 22 and an IDUA-Fc fusion polypeptide having the sequence of SEQ ID NO: 24. The IDUA-Fc fusion protein may also be further processed during cell culture production, such that the TfR-binding modified Fc polypeptide has the sequence of SEQ ID NO: 23 and/or the IDUA-Fc fusion polypeptide has the sequence of SEQ ID NO: 25. Thus, the IDUA-Fc fusion proteins may have unprocessed sequences (i.e., SEQ ID NOs: 22 and 24); one or more processed sequences (i.e., selected from SEQ ID NOs: 23 and 25); or a mixture comprising processed and unprocessed protein molecules.

### Measurement of N-Acetylneuraminic Acid (NeuAc or Sialic Acid) Content

NeuAc (sialic acid) content in the IDUA-Fc fusion proteins was measured by liquid chromatography-mass spectrometry analysis as described in Example 1.

### Example 3. Effect of Sialyltransferase Gene Overexpression and Media Additives on the Production of an Enzyme Fusion Protein

Clonal cells expressing an enzyme fusion protein containing IDUA enzyme (“IDUA-Fc fusion protein”) were used to test the impact of the following on sialic acid levels in the fusion protein: (1) a combination of media additives, and (2) sialyltransferase gene overexpression in the clonal cells.

Sialic Acid Levels were Improved by a Combination of Media Additives and ST6GAL1 Gene Overexpression

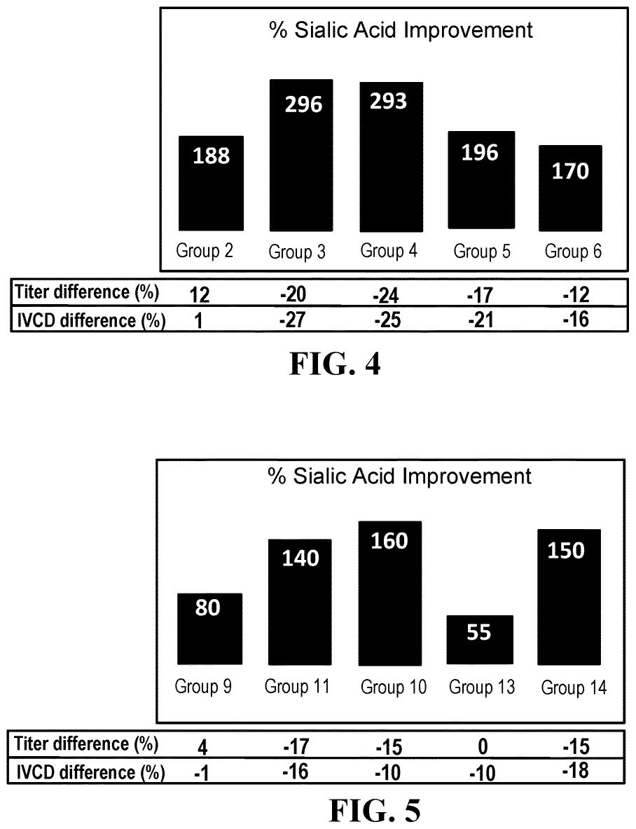

A media additive combination (10 mM ManNAc, 10 μM Mn, and 5 mM uridine) was tested on pools expressing IDUA-Fc fusion proteins alone or in combination with either ST6GAL1 or ST3GAL4 overexpression to determine if sialic acid levels and other product quality attributes were impacted. As illustrated in FIG. 4 and Table 3, ST6GAL1 overexpression enhanced the effect of the media additives on the sialic acid levels of the expressed fusion proteins. The combination of ST6GAL1 overexpression in the presence of media additives increased the sialic acid levels of the fusion protein by at least 290%. In contrast, the presence of media additives alone increased the sialic acid levels over baseline by about 188%. All sialic acid level comparisons were made relative to sialic acid levels in fusion proteins expressed in cells without sialyltransferase gene overexpression and without media additives. Slightly lower titer (about 12-24% decreased titer) was observed in clones expressing sialyltransferase genes.

TABLE 3
Sialic acid level content and product quality in IDUA-Fc fusion proteins

| | | Media Additives | | | | Sialic Acid | |
|---|---|---|---|---|---|---|---|
| | Sialyltransferase | ManNAc | Mn | Uridine | Sialic Acid | Improvement | Titer |
| Group | [Stringency] | (mM) | (μM) | (mM) | (mol/mol) | (%) | (%) |
| 1 | None | 0 | 0 | 0 | 1.28 | | |
| (Control) | | | | | | | |
| 2 | None | 10 | 10 | 5 | 3.69 | 188.3 | 11.7 |
| 3 | ST6GAL1 | 10 | 10 | 5 | 5.07 | 296.1 | −20.1 |
| | [3 μg/mL puromycin] | | | | | | |
| 4 | ST6GAL1 | 10 | 10 | 5 | 5.03 | 293.0 | −24.2 |
| | [4 μg/mL puromycin] | | | | | | |
| 5 | ST3GAL4 | 10 | 10 | 5 | 3.79 | 196.1 | −16.7 |
| | [3 μg/mL puromycin] | | | | | | |
| 6 | ST3GAL4 | 10 | 10 | 5 | 3.45 | 169.5 | −11.8 |
| | [4 μg/mL puromycin] | | | | | | |

### Recombinant Protein Expression and Purification

Stable CHO pool was generated by transfection of transposon constructs encoding IDUA-Fc fusion protein and transposase mRNA into GS-KO CHO-K1 cells (Horizon) by electroporation and subsequent selection in a commercially available, chemically-defined, animal component-free CHO growth medium that is glutamine-free. This pool was subsequently re-transfected with transposon constructs encoding ST3GAL4 (NCBI Accession No. XP_047283377.1) or ST6GAL1 (NCBI Accession No. NP_001340845.1) and transposase mRNA and selected in media with puromycin at different concentrations to control stringency of selection (“supertransfected” cells).

Effect of media additives was evaluated in a 14-day fed-batch production process. On day 0, cells were seeded at about 0.5×10⁶ cells/mL density in a commercially available, chemically-defined, animal component-free CHO growth medium that is glutamine-free. The cultures were maintained at 37° C. in 5% CO₂ and 85% relative humidity. When cultures reached a density of about 10-15×10⁶ cells/mL, they were shifted to 32° C. for the remaining duration of the run. Commercially available feeds were added on days 3, 5, 7, 10 and 12. Media additives were added in 2-3 aliquots distributed over the entire run time. Cultures were harvested on day 14 by centrifugation (5000×g, 15-20 min) followed by sterile filtration through a 0.22 μm filter and stored at 4° C.

IDUA-Fc fusion proteins were purified from cell culture supernatants using Protein A affinity chromatography. Supernatants were loaded onto an equilibrated (PBS) Protein A affinity drip column which was then re-equilibrated before the bound proteins were eluted using a low pH sodium acetate buffer. The eluted proteins were then neutralized using a Tris pH 8 buffer to a final pH of 5.5.

The IDUA-Fc fusion proteins that were expressed and purified are described in Example 2.

### Measurement of N-Acetylneuraminic Acid (NeuAc or Sialic Acid) Content

NeuAc (sialic acid) content in the IDUA-Fc fusion proteins was measured by liquid chromatography-mass spectrometry analysis as described in Example 1.

### Example 4. Effect of Galactosyltransferase and Sialyltransferase Gene Overexpression on the Production of an Enzyme Fusion Protein

Clonal cells expressing an enzyme fusion protein containing IDUA enzyme (“IDUA-Fc fusion protein”) were used to test the impact of the combination of sialyltransferase and galactosyltransferase gene overexpression on sialic acid levels in the fusion protein. Experiments were carried in the presence and absence of media additives (10 mM ManNAc, 10 μM Mn, and 2.5 mM uridine).

Sialic Acid Levels were Improved Primarily by ST6GAL1 Gene Overexpression

Gene overexpression of galactosyltransferase (B4GALT1) was tested in clones expressing IDUA-Fc fusion proteins that were transformed to express sialyltransferases ST6GAL1 and/or ST3GAL4 to determine if sialic acid levels and other product quality attributes of the fusion proteins were impacted. The experiments were carried out in both the presence and absence of a media additive combination (10 mM ManNAc, 10 μM Mn, and 2.5 mM uridine). As illustrated in FIG. 5 and Table 4, B4GALT1 gene overexpression did not appear to impact sialic acid levels of the fusion proteins in clones that also overexpressed ST6GAL1 and/or ST3GAL4. Improvement in sialic acid levels was primarily driven by ST6GAL1 gene overexpression and the presence of the media additives. All sialic acid level comparisons were made relative to sialic acid levels in fusion proteins expressed in cells without galactosyltransferase and sialyltransferase gene overexpression and without media additives.

TABLE 4
Sialic acid level content and product quality in IDUA-Fc fusion proteins

| | | Media Additives | | | | Sialic Acid | |
|---|---|---|---|---|---|---|---|
| | Genes expressed | ManNAc | Mn | Uridine | Sialic Acid | Improvement | Titer |
| Group | [Plasmid ratio] | (mM) | (μM) | (mM) | (mol/mol) | (%) | (%) |
| 1 (Control) | None | 0 | 0 | 0 | 2 | 0 | 0.0 |
| 2 | B4GALT1 + ST6GAL1 | 0 | 0 | 0 | 2.7 | 35 | −20.0 |
| | [1:1] | | | | | | |
| 3 | ST6GAL1 | 0 | 0 | 0 | 3.3 | 65 | −22.1 |
| 4 | B4GALT1 + ST6GAL1 | 0 | 0 | 0 | 3.2 | 60 | −16.7 |
| | [1:3] | | | | | | |
| 5 | B4GALT1 + ST3GAL4 | 0 | 0 | 0 | 2.4 | 20 | −15.6 |
| | [1:1] | | | | | | |
| 6 | B4GALT1 + ST6GAL1 + ST3GAL4 | 0 | 0 | 0 | 2.2 | 10 | −10.4 |
| | [1:1:1] | | | | | | |
| 7 | B4GALT1 + ST6GAL1 + ST3GAL4 | 0 | 0 | 0 | 2.6 | 30 | −11.2 |
| | [1:2:1] | | | | | | |
| 8 | B4GALT1 + ST6GAL1 + ST3GAL4 | 0 | 0 | 0 | 2.4 | 20 | −20.7 |
| | [1:4:1] | | | | | | |
| 9 | None | 10 | 10 | 2.5 | 3.6 | 80 | 4.4 |
| 10 | B4GALT1 + ST6GAL1 | 10 | 10 | 2.5 | 5.2 | 160 | −14.7 |
| | [1:1] | | | | | | |
| 11 | ST6GAL1 | 10 | 10 | 2.5 | 4.8 | 140 | −17.2 |
| 12 | B4GALT1 + ST6GAL1 | 10 | 10 | 2.5 | 4.7 | 135 | −12.8 |
| | [1:3] | | | | | | |
| 13 | B4GALT1 + ST3GAL4 | 10 | 10 | 2.5 | 3.1 | 55 | 0.1 |
| | [1:1] | | | | | | |
| 14 | B4GALT1 + ST6GAL1 + ST3GAL4 | 10 | 10 | 2.5 | 5 | 150 | −14.9 |
| | [1:1:1] | | | | | | |
| 15 | B4GALT1 + ST6GAL1 + ST3GAL4 | 10 | 10 | 2.5 | 5 | 150 | −7.9 |
| | [1:2:1] | | | | | | |
| 16 | B4GALT1 + ST6GAL1 + ST3GAL4 | 10 | 10 | 2.5 | 4.2 | 110 | −14.8 |
| | [1:4:1] | | | | | | |

### Recombinant Protein Expression and Purification

Stable CHO pool was generated by transfection of transposon constructs encoding IDUA-Fc fusion protein and transposase mRNA into GS-KO CHO-K1 cells (Horizon) by electroporation and subsequent selection in a commercially available, chemically-defined, animal component-free CHO growth medium that is glutamine-free. This pool was subsequently re-transfected with transposon constructs encoding B4GALT1 (NCBI Accession No. NP_001488.2), ST3GAL4 (NCBI Accession No. XP_047283377.1), or ST6GAL1 (NCBI Accession No. NP_001340845.1) and transposase mRNA and selected in media with 8 μg/mL puromycin (“supertransfected” cells).

Effect of media additives was evaluated in a 14-day fed-batch production process. On day 0, cells were seeded at about 0.5×10⁶ cells/mL density in a commercially available, chemically-defined, animal component-free CHO growth medium that is glutamine-free. The cultures were maintained at about 37° C. in 5% CO₂ and 85% relative humidity. When cultures reached a density of about 10-15×10⁶ cells/mL, they were shifted to about 32° C. for the remaining duration of the run. Commercially available feeds were added on days 3,5,7,10 and 12. Media additives were added in 2-3 aliquots distributed over the entire run time. Cultures were harvested on day 14 by centrifugation (5000×g, 15-20 min) followed by sterile filtration through a 0.22 μm filter and stored at 4° C.

IDUA-Fc fusion proteins were purified from cell culture supernatants using Protein A affinity chromatography. Supernatants were loaded onto an equilibrated (PBS) Protein A affinity drip column which was then re-equilibrated before the bound proteins were eluted using a low pH sodium acetate buffer. The eluted proteins were then neutralized using a Tris pH 8 buffer to a final pH of 5.5.

The IDUA-Fc fusion proteins that were expressed and purified are as described in Example 2.

### Measurement of N-Acetylneuraminic Acid (NeuAc or Sialic Acid) Content

NeuAc (sialic acid) content in the IDUA-Fc fusion proteins was measured by liquid chromatography-mass spectrometry analysis as described in Example 1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of increasing the sialic acid level in a recombinant glycosylated protein, comprising:
culturing a mammalian host cell capable of expressing the recombinant glycosylated protein in a liquid culture medium comprising one or more media additives selected from the group consisting of: hydrocortisone, N-acetylmannosamine (ManNAc), manganese, and uridine,
wherein the mammalian host cell has been previously transformed with one or more vectors encoding one or more sialyltransferases.

2. The method of claim 1, wherein the method further comprises, prior to the culturing step, transforming the mammalian cell capable of expressing the recombinant glycosylated protein with one or more vectors encoding the one or more sialyltransferases to generate the mammalian host cell.

3. The method of claim 1 or 2, wherein the mammalian host cell is a rodent cell.

4. The method of claim 3, wherein the rodent cell is a CHO cell.

5. The method of claim 1 or 2, wherein the mammalian host cell is a human cell.

6. The method of claim 1 or 2, wherein the one or more sialyltransferases are selected from the group consisting of: ST6GAL1 and ST3GAL4.

7. The method of claim 6, wherein the one or more vectors encode ST6GAL1.

8. The method of any one of claims 1-7, wherein the liquid culture medium comprises one of: hydrocortisone, N-acetylmannosamine (ManNAc), manganese, or uridine.

9. The method of any one of claims 1-7, wherein the liquid culture medium comprises two of: hydrocortisone, N-acetylmannosamine (ManNAc), manganese, and uridine.

10. The method of any one of claims 1-7, wherein the liquid culture medium comprises three of: hydrocortisone, N-acetylmannosamine (ManNAc), manganese, and uridine.

11. The method of any one of claims 1-7, wherein the liquid culture medium comprises hydrocortisone, N-acetylmannosamine (ManNAc), manganese, and uridine.

12. The method of any one of claims 1-11, wherein the ManNAc is present in the liquid culture medium at a concentration of from 10 mM to about 60 mM.

13. The method of any one of claims 1-12, wherein the uridine is present in the liquid culture medium at a concentration of from 2.5 mM to about 10 mM.

14. The method of any one of claims 1-13, wherein the hydrocortisone is present in the liquid culture medium at a concentration of from 5 μM to about 50 μM.

15. The method of any one of claims 1-14, wherein the manganese is present in the liquid culture medium at a concentration of from 2 μM to about 15 M.

16. The method of any one of claims 1-15, wherein the recombinant glycosylated protein is a recombinant glycosylated enzyme.

17. The method of claim 16, wherein the recombinant glycosylated enzyme comprises an enzyme replacement therapy (ERT) enzyme, a catalytically active ERT enzyme variant, or a catalytically active ERT enzyme fragment.

18. The method of claim 17, wherein the enzyme replacement therapy (ERT) enzyme comprises a lyosomal storage disease (LSD) enzyme.

19. The method of claim 18, wherein the LSD enzyme is alpha-L-iduronidase (IDUA) or N-sulfoglucosamine sulfohydrolase (SGSH).

20. The method of claim 16, wherein the recombinant glycosylated enzyme is a fusion protein.

21. The method of claim 20, wherein the fusion protein comprises (i) a first Fc polypeptide that is linked to an enzyme replacement therapy (ERT) enzyme, an ERT enzyme variant, or a catalytically active fragment thereof; and (ii) a second Fc polypeptide that forms an Fc dimer with the first Fc polypeptide, wherein the first Fc polypeptide and/or the second Fc polypeptide is a modified Fc polypeptide capable of specifically binding to a transferrin receptor (TfR).

22. The method of claim 20, wherein the fusion protein comprises (i) an enzyme replacement therapy (ERT) enzyme, an ERT enzyme variant, or a catalytically active fragment thereof; and (ii) a modified Fc dimer that is capable of specifically binding to a transferrin receptor (TfR).

23. The method of claim 20, wherein the fusion protein comprises an enzyme replacement therapy (ERT) enzyme, an ERT enzyme variant, or a catalytically active fragment thereof, linked to an Fc polypeptide.

24. The method of claim 23, wherein the Fc polypeptide is a modified Fc polypeptide.

25. The method of claim 24, wherein the Fc polypeptide is capable of specifically binding to a transferrin receptor (TfR).

26. The method of any one of claims 1-25, wherein the culturing is fed-batch culturing.

27. The method of any one of claims 1-25, wherein the culturing is batch culturing.

28. The method of any one of claims 1-25, wherein the culturing is perfusion culturing.

29. The method of any one of claims 1-28, wherein the sialic acid level in the recombinant glycosylated protein is increased by at least 50% as compared to the recombinant glycosylated protein produced by a method that does not include one or both of (i) the use of a mammalian host cell capable of expressing the recombinant glycosylated protein comprising a vector encoding the one or more sialyltransferases, and (ii) culturing a mammalian host cell capable of expressing the recombinant glycosylated protein in a liquid culture medium comprising one or more media additives selected from the group consisting of: hydrocortisone, N-acetylmannosamine (ManNAc), manganese, and uridine.

30. The method of any one of claims 1-28, wherein the sialic acid level in the recombinant glycosylated protein is increased by at least 100% as compared to the recombinant glycosylated protein produced by a method that does not include one or both of (i) the use of a mammalian host cell capable of expressing the recombinant glycosylated protein comprising a vector encoding the one or more sialyltransferases, and (ii) culturing a mammalian host cell capable of expressing the recombinant glycosylated protein in a liquid culture medium comprising one or more media additives selected from the group consisting of: hydrocortisone, N-acetylmannosamine (ManNAc), manganese, and uridine.

31. The method of any one of claims 1-28, wherein the sialic acid level in the recombinant glycosylated protein is increased by at least 150% as compared to the recombinant glycosylated protein produced by a method that does not include one or both of (i) the use of a mammalian host cell capable of expressing the recombinant glycosylated protein comprising a vector encoding the one or more sialyltransferases, and (ii) culturing a mammalian host cell capable of expressing the recombinant glycosylated protein in a liquid culture medium comprising one or more media additives selected from the group consisting of: hydrocortisone, N-acetylmannosamine (ManNAc), manganese, and uridine.

32. The method of any one of claims 1-28, wherein the sialic acid level in the recombinant glycosylated protein is increased by at least 250% as compared to the recombinant glycosylated protein produced by a method that does not include one or both of (i) the use of a mammalian host cell capable of expressing the recombinant glycosylated protein comprising a vector encoding the one or more sialyltransferases, and (ii) culturing a mammalian host cell capable of expressing the recombinant glycosylated protein in a liquid culture medium comprising one or more media additives selected from the group consisting of: hydrocortisone, N-acetylmannosamine (ManNAc), manganese, and uridine.

33. The method of any one of claims 1-32, wherein the method further comprises harvesting the recombinant glycosylated protein from the liquid culture medium and/or the mammalian host cell.

34. The method of claim 33, wherein the method further comprises isolating the harvested recombinant glycosylated protein.

35. The method of claim 34, wherein the method further comprises formulating the isolated recombinant glycosylated protein.

36. A recombinant glycosylated protein produced by the method of any one of claims 1-35.

37. A method of treating a subject in need thereof comprising administering to the subject a therapeutically effective amount of the recombinant glycosylated protein of claim 36.
